(19) United States
(12) Patent Application Publication
(10) Pub. No.: US 2021/0304034 A1
(43) Pub. Date: Sep. 30, 2021

(54) LIFESTYLE HABIT RECOMMENDATION APPARATUS, AND LIFESTYLE HABIT RECOMMENDATION METHOD

(71) Applicant: NEC Solution Innovators, Ltd., Tokyo (JP)

(72) Inventor: Jou AKITOMI, Tokyo (JP)

(73) Assignee: NEC Solution Innovators, Ltd., Tokyo (JP)

(21) Appl. No.: 17/210,597

(22) Filed: Mar. 24, 2021

(30) Foreign Application Priority Data
Mar. 24, 2020 (JP) 2020-053133

(51) Int. Cl. G06N 5/04; G06N 20/00

(52) U.S. Cl. CPC G06N 5/04; G06N 20/00

(57) ABSTRACT

A lifestyle habit recommendation apparatus 10 of the present invention, includes: an execution information acquisition unit 11; a vitality level calculation unit 12; a recommended information determination unit 13; and an output unit 14, wherein the execution information acquisition unit 11 acquires execution information executed for each item serving as an indicator of a lifestyle habit and a vitality level of a user in association with a date, the vitality level calculation unit 12 calculates, based on the execution information and the vitality level of the user in a predictive input period, the vitality level of the user after a predetermined number of days from the predictive input period, the recommended information determination unit 13 calculates recommended execution information up to the predetermined number of days so that the calculated vitality level can be raised, and the output unit 14 outputs the recommended execution information to the user.

### CROSS-REFERENCE TO RELATED APPLICATION

This application claims priority from Japanese Patent Application No. 2020-053133 filed on Mar. 24, 2020. The entire subject matter of the Japanese Patent Application is incorporated herein by reference.

### TECHNICAL FIELD

The present invention relates to a lifestyle habit recommendation apparatus, a lifestyle habit recommendation method, and a program.

### BACKGROUND ART

Cognitive Behavioral Therapy for Insomnia (CBT-I) is known as an effective treatment modality for insomnia. CBT-I is a psychotherapy that aims to control sleep by reviewing sleep-related cognitive and behavioral habits.

Against this background, an application for recording a sleep habit has been provided (WO2019/035166). According to this application, it is possible to manage and improve the sleep habit of the user.

### SUMMARY OF INVENTION

### Technical Problem

WO2019/035166 discloses the invention that analyzes what has been a hindrance of sleep and generates advice information for eliminating the same, based on data obtained by a user using the application. WO2019/035166, however, does not disclose the invention that predicts the vitality level after a predetermined number of days and recommends an optimal sleep habit so that the vitality level can be raised. Such problems are not limited to a sleep habit but are common to various lifestyle habits including a sleep habit.

With the foregoing in mind, it is an object of the present invention to provide a system which predicts a vitality level after a predetermined number of days and recommends an optimum lifestyle habit so that the vitality level can be raised.

### Solution to Problem

In order to achieve the above object, the present invention provides a lifestyle habit recommendation apparatus, including: an execution information acquisition unit; a vitality level calculation unit; a recommended information determination unit; and an output unit, wherein the execution information acquisition unit acquires execution information executed for each item serving as an indicator of a lifestyle habit and a vitality level of a user in association with a date, the vitality level calculation unit calculates, based on the execution information and the vitality level of the user in a predictive input period, the vitality level of the user after a predetermined number of days from the predictive input period, the recommended information determination unit calculates recommended execution information up to the predetermined number of days so that the calculated vitality level can be raised, and the output unit outputs the recommended execution information to the user.

The present invention also provides a lifestyle habit recommendation method, including the steps of: acquiring execution information; calculating vitality level; determining recommended information; and outputting, wherein the execution information-acquiring acquires execution information executed for each item serving as an indicator of a lifestyle habit and a vitality level of a user in association with a date, the vitality level-calculating calculates, based on the execution information and the vitality level of the user in a predictive input period, the vitality level of the user after a predetermined number of days from the predictive input period, the recommended information-determining calculates recommended execution information up to the predetermined number of days so that the calculated vitality level can be raised, and the outputting outputs the recommended execution information to the user.

### Advantageous Effects of Invention

According to the present invention, it is possible to provide a system which predicts a vitality level after a predetermined number of days and recommends an optimum lifestyle habit so that the vitality level can be raised.

### BRIEF DESCRIPTION OF DRAWINGS

FIG. 1 is a block diagram showing a configuration of an example of a lifestyle habit recommendation apparatus according to the first example embodiment.

FIG. 2 is a block diagram showing an example of the hardware configuration of the lifestyle habit recommendation apparatus according to the first example embodiment.

FIGS. 3A and 3B are diagrams showing an example of a screen output on a terminal of a user in the first example embodiment.

FIG. 4 is a flowchart showing an example of the processing in the lifestyle habit recommendation apparatus according to the first example embodiment.

FIGS. 5A and 5B are diagrams showing an example of determining recommended execution information based on execution information and a vitality level of a user in the first example embodiment.

FIG. 6 is a flowchart showing an example of the processing in a lifestyle habit recommendation apparatus according to the second example embodiment.

FIG. 7 is a diagram showing an example of determining recommended execution information based on execution information and a vitality level of a user in the second example embodiment.

### DESCRIPTION OF EMBODIMENTS

Example embodiments of the present invention will be described. The present invention is not limited to the following example embodiments. In the following drawings, identical parts are indicated with identical reference signs. In addition, the descriptions of the respective example embodiments can be referred to each other unless otherwise specified. Furthermore, the configurations of the example embodiments can be combined unless otherwise specified.

In the present invention, the lifestyle habit is not particularly limited, and examples thereof include sleep habits, work habits (overtime, early leaving, etc.), study habits (study hours, study start time, etc.), exercise habits, and eating habits. The lifestyle habit may be, for example, a daily habit or a habit in any other period.

### First Example Embodiment

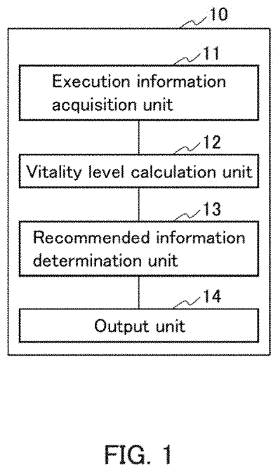

The present example embodiment will be described with reference to a case where the lifestyle habit is a sleep habit and daily habits are managed as an example. The present invention, however, is not limited thereto. FIG. 1 is a block diagram showing the configuration of an example of a lifestyle habit recommendation apparatus 10 of the present example embodiment. The lifestyle habit recommendation apparatus 10 includes an execution information acquisition unit 11, a vitality level calculation unit 12, a recommended information determination unit 13, and an output unit 14. The lifestyle habit recommendation apparatus 10 is also referred to as a lifestyle habit recommendation system, for example.

The lifestyle habit recommendation apparatus 10 may be, for example, a single lifestyle habit recommendation apparatus including the above-described components, or may be a lifestyle habit recommendation apparatus to which the components are connectable via a communication network. The lifestyle habit recommendation apparatus 10 may be, for example, a terminal in which the program of the present invention is installed. Examples of the terminal include a mobile phone, a smart phone, a tablet, and a personal computer (PC). The lifestyle habit recommendation apparatus 10 includes, for example, a terminal and a server, and the terminal and the server may be connectable via a communication network. Examples of the communication network include an Internet line, a telephone line, a local area network (LAN), and a wireless fidelity (WiFi).

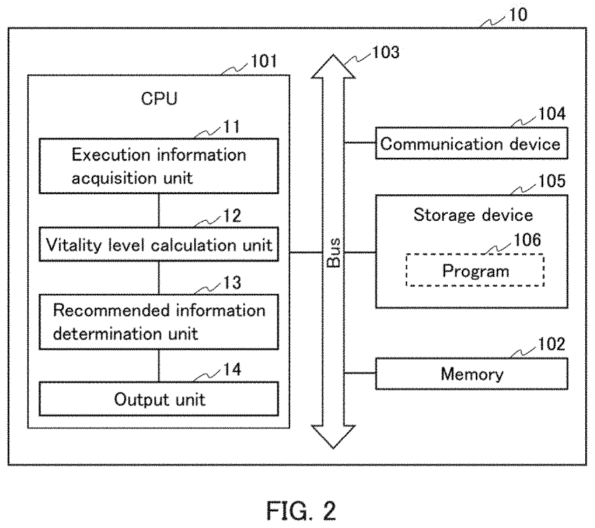

FIG. 2 show a block diagram of the hardware configuration of the lifestyle habit recommendation apparatus 10. The lifestyle recommendation apparatus 10 includes, for example, a central processing unit (CPU) 101, a memory 102, a bus 103, a communication device 104, a storage device 105, and the like. The components of the lifestyle habit recommendation apparatus 10 are connected to each other via a bus 103 by, for example, respective interfaces.

The CPU101 serves to control the entire lifestyle habit recommendation apparatus 10. In the lifestyle habit recommendation apparatus 10, the CPU 101 executes a program of the present invention and other programs, and reads and writes various pieces of information, for example. Specifically, for example, the CPU101 functions as the execution information acquisition unit 11, the vitality level calculation unit 12, the recommended information determination unit 13, and the output unit 14.

The bus 103 can also be connected to an external device, for example. Examples of the external device include a terminal, an external storage device (such as an external database), and a printer. The lifestyle habit recommendation apparatus 10 can be connected to a communication network by, for example, a communication device 104 connected to the bus 103, and can also be connected to the external device via the communication network.

The memory 102 includes, for example, a main memory, and the main memory is also referred to as a main storage device. When the CPU101 performs processing, the memory 102 reads various kinds of operation programs such as the program of the present invention stored in a storage device 105 to be described below, and the CPU101 receives data from the memory 102 and executes the program. The main memory is, for example, a random access memory (RAM). The memory 102 further includes, for example, a read-only memory (ROM).

The storage device 105 is also referred to as, for example, a so-called auxiliary storage device with respect to the main memory (main storage device). As described above, the storage device 105 stores the operation program 106 including the program of the present invention. The storage device 105 includes, for example, a storage medium and a drive for reading from and writing to the storage medium. The storage medium is not particularly limited, and may be, for example, a built-in type or an external type, and examples thereof include a hard disk (HD), a Floppy® disk (FD), a CD-ROM, a CD-R, a CD-RW, an MO, a DVD, a flash memory, and a memory card. The drive is not particularly limited. The storage device 105 may be, for example, a hard disk drive (HDD) in which the storage medium and the drive are integrated. For example, the storage device 105 stores the operation program 106 as described above. Further, the storage device 105 may store, for example, execution information and information such as a vitality level, which will be described below.

The lifestyle habit recommendation apparatus 10 may further include, for example, an input device and an output device such as a display. Examples of the input device include a touch panel, a keyboard, and a mouse. Examples of the display include an LED display and a liquid crystal display.

In the lifestyle habit recommendation apparatus 10, the memory 102 and the storage device 105 may also store access information and log information from the user and information acquired from an external database (not shown).

The execution information acquisition unit 11 acquires execution information executed for each item serving as an indicator of a lifestyle habit and a vitality level of a user in association with a date. The date is, for example, the year, month, and day. The present invention, however, is not limited thereto, and the date may be the number of days elapsed from a given set date or the like.

In the execution information, the items to be the indicators of the sleep habit are not particularly limited, and examples thereof include a record of sleep and supplementary information related to sleep, and specific examples thereof include sleep hours (actual sleep hours), getting-into-bed time (time of getting into bed), falling-asleep-time (time of falling asleep), awaking time (time of awaking), getting-out-of-bed-time (time of getting out of bed), arousal during sleep, sleep efficiency (ratio of sleep hours to bedtime), behavior before sleeping, food and drink (alcohol, coffee, tobacco, etc.), and whether or not to take a nap and its time. The execution information may include one or two ore more of the items.

For example, evaluation criteria may be set for the items. The specific content of the item can be evaluated based on the evaluation criteria. The evaluation criteria may be set in advance, for example, based on a value which is generally considered to be good. Specifically, the evaluation criteria may be set such that the average sleep hours in the range of 7 to 8 hours is evaluated as good, alcohol intake before sleeping is evaluated as poor, or the like, for example.

The execution information may further include auxiliary information for using for calculation of the vitality level to be described below, for example. The auxiliary information includes, for example, personal attribute data of the user, and specific examples thereof include a chief complaint (difficulty in sleeping, arousal during sleep, waking in the early morning, feeling of drowsiness, and the number thereof), a score of Athens Insomnia Scale (AIS), sex, and age.

The vitality level is a value indicating the vitality level of the user and a value reflecting the vitality level of the user, and is not particularly limited, and, for example, may be based on the subjectivity of the user, may be a value (a biological value or the like) measured by a sensor or the like, may be a result of a given test capable of measuring the vitality level, may be a degree of achievement of a predetermined target, or the like. The vitality level may be, for example, a parameter indicating the vigor level, performance, concentration, motivation, awareness (less sleepiness), productivity, tone of the skin, how vigorous it was on that day, and the like. The vitality level is, for example, a value indicating the vitality level during the day of the user. The vitality level may have, for example, 3 stages, 5 stages, or the like.

The criteria for the vitality level may be set in advance or may be set freely by the user, for example. For example, in the case where the vitality level has 5 stages, the vitality level can be determined to be “low” when the vitality level is “1” or “2”, the vitality level can be determined to be “ordinary” when the vitality level is “3”, and the vitality level can be determined to be “high” when the vitality level is “4” or “5”.

The execution information and the vitality level may be acquired based on input by the user or may be acquired directly from the sensor or the like, for example. The execution information acquisition unit 11 may calculate the execution information based on the acquired data, for example. As the execution information and the vitality level data, for example, the accumulated data can be used by the user managing the sleep habit using the lifestyle habit recommendation apparatus 10.

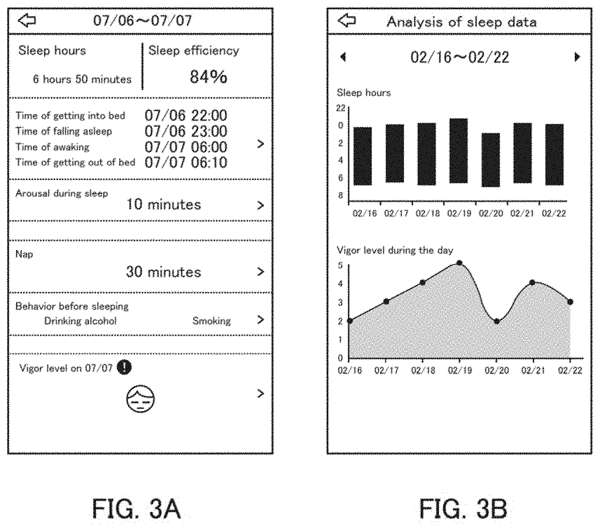

FIGS. 3A and 3B show an example of a display screen on a terminal of the user. In FIG. 3A, based on the input to the terminal of the user, as the execution information of July 6 to July 7, the sleep hours, the sleep efficiency, the time of getting into bed, the time of falling asleep, the time of awaking, the time of getting out of bed, the arousal during sleep, the nap hours, and the food and drink taken before sleeping are displayed, and as the vitality level on July 7, the vigor level during the day is displayed (in FIG. 3A, the vigor level is scored in five stages and displayed by the expression of the character). In FIG. 3B, the sleep hours is displayed as the execution information from February 16 to February 22, and the vigor level during the day is displayed as the vitality level.

The vitality level calculation unit 12 calculates, based on the execution information and the vitality level of the user in a predictive input period, the vitality level of the user after a predetermined number of days from the predictive input period. The predictive input period and the predetermined number of days are not particularly limited, and can be set based on, for example, input by the user. The predictive input period may be, for example, 3 to 10 days, 1 week, etc. The predetermined number of days may be referred to as, for example, “near future” and may be several days, and may be specifically, for example, 1 day (the day following the predictive input period) to 7 days, 2 days to 7 days, 2 days, or 3 days.

For example, the vitality level calculation unit 12 can calculate the vitality level based on a predetermined calculation method. Specifically, for example, the execution information of the user in the predictive input period can be evaluated based on the evaluation criteria, and the vitality level can be calculated based on the evaluation.

Further, in the calculation of the vitality level, the vitality level calculation unit 12 may use sample information including the execution information and the vitality level acquired in association with a date, for example. The sample information may be, for example, data of the user or of others. The sample information may be data in the predictive input period or data in a period other than the predictive input period. The sample information may be data of one person or data of two or more persons. Specifically, for example, first, data similar to the execution information and the vitality level of the user in the predictive input period is extracted from the execution information and the vitality level in the sample information by a known method (e.g., method using correlation between feature vectors). Then, the vitality level after the predetermined number of days from the period associated with the extracted execution information and the vitality level in the sample information is acquired. The acquired vitality level may be the vitality level of the user after a predetermined number of days from the predictive input period.

In the calculation of the vitality level, the vitality level calculation unit 12 may use, for example, a learned model in which the execution information and the vitality level in the predictive input period are regarded as input and the vitality level after a predetermined number of days from the predictive input period is regarded as output. The learned model is to be described below.

The recommended information determination unit 13 calculates recommended execution information up to the predetermined number of days so that the calculated vitality level can be raised.

For example, the recommended information determination unit 13 can calculate the recommended execution information using the execution information in the predictive input period. Specifically, for example, the execution information (e.g., average value) of the user in the predictive input period can be changed so that the evaluation based on the evaluation criteria can be raised, and this can be used as the recommended execution information. Specifically, for example, when the average sleep hours is less than 7 hours, sleep hours of 7 hours or more is recommended, alcohol intake before sleeping is recommended, and the like.

In the calculation of the recommended execution information, the recommended information determination unit 13 may use, for example, a learned model in which the vitality level is regarded as input and the recommended execution information is regarded as output. In this case, the vitality level to be input may be a value higher than the calculated vitality level.

For example, the recommended information determination unit 13 may determine whether or not the calculated vitality level is low, and may calculate the recommended execution information when the vitality level is determined to be low. Thereby, the health action for avoiding the condition (bad condition) in which the vitality level is low can be recommended to the user. The user can spend healthy days by avoiding a bad condition in the future. Furthermore, by recognizing that a bad condition can be avoided, a good effect on the psychological aspect can be expected.

The output unit 14 outputs the recommended execution information to the user. The output recommended execution information may be transmitted to the terminal of the user via the communication device 104, may be displayed on the display or the like of the lifestyle habit recommendation apparatus 10, or may be output on a file, for example.

The output unit 14 may further output a notification, for example. For example, when the vitality level calculated by the vitality level calculation unit 12 is low, the notification that the vitality level is low (e.g., “Tomorrow seems to be less vigorous!”) is given as the notification. This can motivate the user to improve the lifestyle habit.

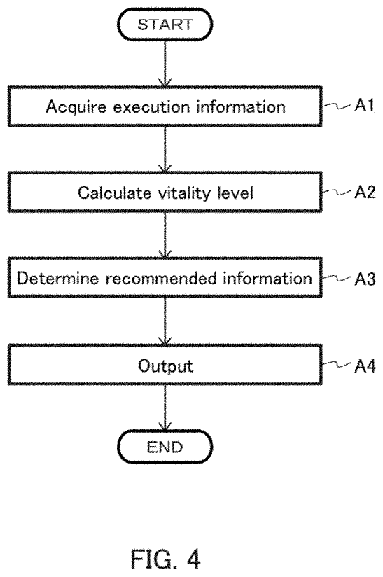

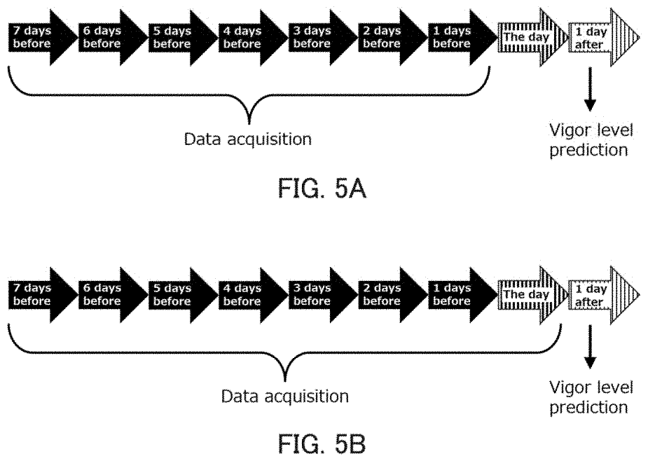

Next, the lifestyle habit recommendation method of the present example embodiment will be described with reference to FIGS. 4, 5A, and 5B. FIG. 4 is a flowchart showing an example of the lifestyle habit recommendation method. FIGS. 5A and 5B are diagrams showing an example of determining the recommended execution information based on the execution information and the vitality level of the user. The lifestyle habit recommendation method of the present example embodiment can be implemented as follows using, for example, the lifestyle habit recommendation apparatus 10 of FIG. 1. The lifestyle habit recommendation method of the present example embodiment is not limited to the use of the lifestyle habit recommendation apparatus 10 of FIG. 1.

First, the execution information acquisition unit 11 acquires execution information executed for each item serving as an indicator of a lifestyle habit and a vitality level of a user in association with a date (step (A1)). For example, as shown in FIG. 5A, the execution information (sleep data) and the vitality level of the user are acquired on each date of 1 day to 7 days before “the day”, which correspond to the predictive input period. In FIG. 5A, “the day” indicates the day on which the vitality level is calculated by the following step (A2) and the recommended execution information is calculated and output by the following steps (A3) to (A4).

Next, the vitality level calculation unit 12 calculates, based on the execution information and the vitality level of the user in a predictive input period, the vitality level of the user after a predetermined number of days from the predictive input period (step (A2)). In the example of FIG. 5A, the vigor level as the vitality level is calculated 1 day after “the day” (2 days after the predictive input period).

Next, the recommended information determination unit 13 calculates recommended execution information up to the predetermined number of days so that the calculated vitality level can be raised (step (A3)). In the example of FIG. 5A, the recommended execution information (sleep data) of “the day” is calculated.

Next, the output unit 14 outputs the recommended execution information to the user (step (A4)), and ends the procedure (END).

In the example of FIG. 5A, the vitality level after 2 days from the predictive input period is calculated. The present invention, however, is not limited thereto, and as shown in FIG. 5B, the vitality level after 1 day from the predictive input period may be calculated in the step (A2), for example. In this case, for example, as shown in FIG. 5B, in the step (A1), data including the sleep data of the user is acquired as the execution information on each date of 1 day to 7 days before “the day”. Then, on “the day”, data (the number of cigarettes, etc.) of the execution information other than the sleep data of the user is acquired. Next, in the step (A2), the vitality level after 1 day from “the day” (after 1 day from the predictive input period) is calculated. Next, in the steps (A3) and (A4), the recommended execution information (data including sleep data) up to 1 day after “the day” is calculated and output.

### Second Example Embodiment

Next, the second example embodiment will be described. In the present example embodiment, the vitality level calculation unit 12 further acquires provisional execution information of the user up to a predetermined number of days after the predictive input period, and calculates the vitality level of the user based on the provisional execution information and the execution information and the vitality level in the predictive input period. The provisional execution information is the execution information that has not been executed up to a predetermined number of days after the predictive input period. The recommended information determination unit 13 further calculates the recommended execution information using the provisional execution information. The second example embodiment is the same as the first example embodiment other than this point.

The vitality level calculation unit 12 may acquire, for example, a preset value (e.g., an average value of all users) as the provisional execution information, or may calculate the provisional execution information based on the execution information that has already been executed. The execution information that has already been executed may include, for example, the execution information in the predictive input period. When the calculation is performed, the calculation may be performed based on a predetermined calculation method, for example. Specifically, for example, an average value, a mode value, and the like of the execution information in the predictive input period can be used. The calculation may be performed using a learned model in which the execution information in the predictive input period is regarded as input and the provisional execution information after a predetermined number of days from the predictive input period is regarded as output.

In the calculation of the recommended execution information, for example, the recommended information determination unit 13 may change the provisional execution information so that the evaluation based on the evaluation criteria can be raised, and may use the changed provisional execution information as the recommended execution information. Regarding the change, when there are two or more items in the provisional execution information, one of the items may be changed, or some or all of the items may be changed.

In the calculation of the recommended execution information, for example, the recommended information determination unit 13 may first calculate the post-change provisional execution information in which the content of the item in the provisional execution information is changed, for example. In this case, the change may be, for example, a random change. The post-change provisional execution information may be calculated as the recommended execution information when the vitality level calculated using the post-change provisional execution information is higher than the vitality level calculated using the provisional execution information and/or higher than a predetermined threshold.

Specifically, for example, if there are five items in the provisional execution information, it is possible to use the post-change provisional execution information of the combination of 2⁵=32, whether or not to improve the items. For example, the post-change provisional execution information having the highest vitality level may be used as the recommended execution information, or the post-change provisional execution information having the smallest number of items whose contents are to be changed may be used as the recommended execution information. Further, multiple pieces of the recommended execution information may be output in such a manner that they can be displayed in these order. Further, for example, when a result showing the improvement of the vitality level is not obtained in the above 32 kinds of results, information of improving all the items may be used as the recommended execution information.

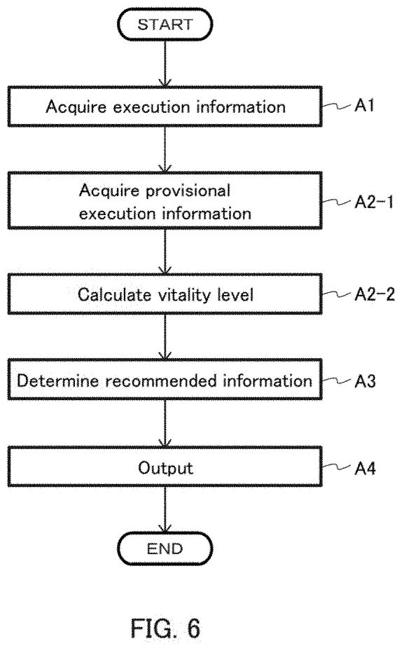

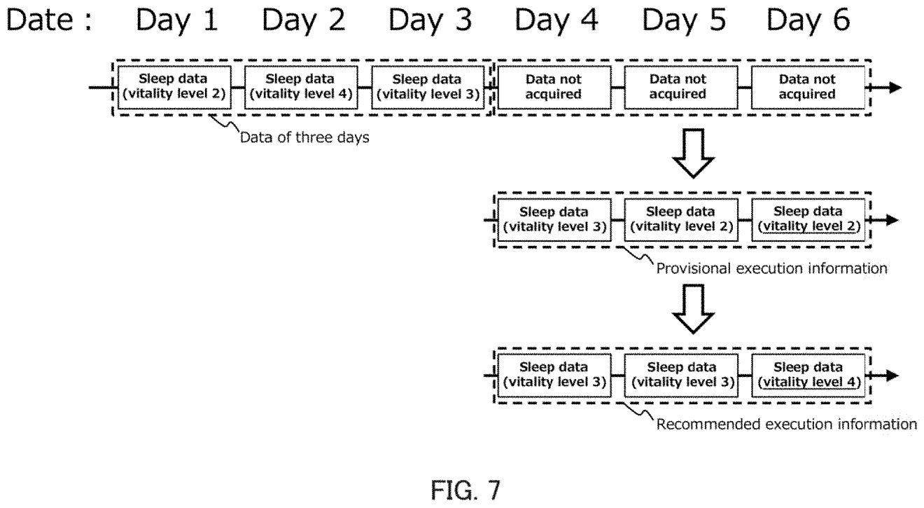

Next, the lifestyle habit recommendation method of the present example embodiment will be described with reference to FIGS. 6 and 7. FIG. 6 is a flowchart showing an example of the lifestyle habit recommendation method. FIG. 7 is a diagram showing an example of determining the recommended execution information based on the execution information and the vitality level of the user.

First, the execution information acquisition unit 11 acquires execution information executed for each item serving as an indicator of a lifestyle habit and a vitality level of a user in association with a date (step (A1)). For example, as shown in FIG. 7, the execution information (sleep data) and the vitality level of the user are acquired on each date of day 1 to day 3.

Next, the vitality level calculation unit 12 acquires a provisional execution information of the user up to a predetermined number of days after the predictive input period (step (A2-1)). In the example of FIG. 7, day 1 to day 3 are in the predictive input period, and the provisional execution information (sleep data) is calculated on each date of day 4 to day 6.

Next, the vitality level calculation unit 12 calculates the vitality level of the user after a predetermined number of days from the predictive input period based on the provisional execution information and the execution information and the vitality level of the user in the predictive input period (step (A2-2)). In the example of FIG. 7, “2” is calculated as the vitality level on the day 6, which is after the predetermined number of days (in this case, after 3 days), based on the provisional execution information and the execution information and the vitality level on each date of day 1 to day 3. In the example of FIG. 7, “3” and “2” are calculated as the vitality levels even on the day 4 and day 5, which are within “after the predetermined number of days”, respectively.

Next, the recommended execution information determination unit 13 calculates recommended execution information up to the predetermined number of days by using the provisional execution information so that the calculated vitality level can be raised (step (A3)). In the example of FIG. 7, the recommended execution information (sleep data) is calculated on day 4 to day 6. In the example of FIG. 7, “4” is calculated as the vitality level when the user executes the recommended execution information on day 6, and which shows that the vitality level when the user executes the recommended execution information is higher than the vitality level when the user executes the provisional execution information.

Next, the output unit 14 outputs the recommended execution information to the user (step (A4)), and ends the procedure (END).

### Third Example Embodiment

The present example embodiment relates to a learned model and a learned model generation method.

The learned model (also referred to as a discriminator) of the present example embodiment includes an input layer, an output layer, and an intermediate layer. The input layer inputs execution information executed for each item serving as an indicator of a lifestyle habit and a vitality level in a predictive input period of a user, the output layer outputs the vitality level of the user after a predetermined number of days from the predictive input period, and the intermediate layer learns parameters by using teacher data in which the execution information and the vitality level acquired in association with the date are regarded as input and the vitality level after a predetermined number of days from the date is regarded as output. The learned model of the present example embodiment causes a computer to function such that the input layer inputs the execution information and the vitality level in the predictive input period, the intermediate layer performs computation, and the output layer outputs the vitality level after a predetermined number of days from the predictive input period.

The learned model may be generated based on the execution information and the vitality level information acquired in association with the date, for example, according to known methods of machine learning such as a support vector machine (SVM), a neural network, and the like. As the learned model, for example, a random forest algorithm can be used.

The learned model generation method of the present example embodiment includes the steps of: acquiring teacher data; and generating learned model, wherein the teacher data-acquiring acquires teacher data including execution information executed for each item serving as an indicator of a lifestyle habit and a vitality level which are acquired in association with a date, and the learned model-generating generates a learned model, in which the execution information and the vitality level of the user in a predictive input period of are regarded as input and the vitality level of the user after a predetermined number of days from the predictive input period is regarded as output, by using the acquired teacher data.

The learned model generation method may further include, for example, the steps of: acquiring provisional execution information; and calculating a vitality level, wherein the provisional execution information-acquiring may acquire provisional execution information of the user up to a predetermined number of days after the predictive input period, and the vitality level-calculating may calculate the vitality level of the user after a predetermined number of days from the predictive input period based on the provisional execution information and the execution information and the vitality level in the predictive input period using the generated learned model. The provisional execution information is as described above. The teacher data-acquiring may acquire the execution information, the provisional execution information, and the calculated vitality level as the teacher data. As described above, by using the provisional execution information, for example, the amount of the teacher data can be increased in the learned model-generating.

The learned model generation method may further include, for example, the steps of acquiring executed information; and modifying, wherein the executed information-acquiring acquires executed information of the user, the executed information may include the vitality level after a predetermined number of days from the predictive input period, and the modifying may modify a computation parameter of the learned model such that the executed information matches the vitality level output by the learned model. Thus, for example, the learned model with higher accuracy can be generated.

### Fourth Example Embodiment

The program of the present example embodiment is a program for causing a computer to execute at least one of the lifestyle habit recommendation method and the learned model generation method of the aforementioned example embodiments as a procedure. In the present invention, the “procedure” may be read as “processing”. The program of the present example embodiment may be recorded on, for example, a computer readable recording medium. The recording medium is not particularly limited, and examples thereof include a read-only memory (ROM), a hard disk (HD), and an optical disk.

While the present invention has been described above with reference to illustrative example embodiments, the present invention is by no means limited thereto. Various changes and variations that may become apparent to those skilled in the art may be made in the configuration and specifics of the present invention without departing from the scope of the present invention.

(Supplementary Notes)

Some or all of the above example embodiments and examples may be described as in the following Supplementary Notes, but are not limited thereto.

### (Supplementary Note 1)

A lifestyle habit recommendation apparatus, including:

an execution information acquisition unit;

a vitality level calculation unit;

a recommended information determination unit; and

an output unit, wherein

the execution information acquisition unit acquires execution information executed for each item serving as an indicator of a lifestyle habit and a vitality level of a user in association with a date,

the vitality level calculation unit calculates, based on the execution information and the vitality level of the user in a predictive input period, the vitality level of the user after a predetermined number of days from the predictive input period,

the recommended information determination unit calculates recommended execution information up to the predetermined number of days so that the calculated vitality level can be raised, and

the output unit outputs the recommended execution information to the user.

### (Supplementary Note 2)

The lifestyle habit recommendation apparatus according to Supplementary Note 1, wherein

the recommended information determination unit calculates the recommended execution information when the calculated vitality level is low.

### (Supplementary Note 3)

The lifestyle habit recommendation apparatus according to Supplementary Note 1 or 2, wherein

the vitality level calculation unit calculates the vitality level of the user using sample information including the execution information and the vitality level acquired in association with the date.

### (Supplementary Note 4)

The lifestyle habit recommendation apparatus according to any one of Supplementary Notes 1 to 3, wherein

the vitality level calculation unit calculates the vitality level of the user using a learned model in which the execution information and the vitality level in the predictive input period are regarded as input and the vitality level after a predetermined number of days from the predictive input period is regarded as output.

### (Supplementary Note 5)

The lifestyle habit recommendation apparatus according to any one of Supplementary Notes 1 to 4, wherein

the vitality level calculation unit further acquires provisional execution information of the user up to a predetermined number of days after the predictive input period, and calculates the vitality level of the user based on the provisional execution information and the execution information and the vitality level in the predictive input period.

### (Supplementary Note 6)

The lifestyle habit recommendation apparatus according to Supplementary Note 5, wherein

the vitality level calculation unit calculates the provisional execution information based on the execution information in the predictive input period.

### (Supplementary Note 7)

The lifestyle habit recommendation apparatus according to Supplementary Note 5 or 6, wherein

the vitality level calculation unit further calculates the vitality level of the user based on post-change provisional execution information and the execution information and the vitality level in the predictive input period,

the post-change provisional execution information is obtained by changing a content of the item serving as an indicator of a lifestyle habit in the provisional execution information, and

the recommended information determination unit calculates the post-change provisional execution information as the recommended execution information when the vitality level calculated using the post-change provisional execution information is higher than the vitality level calculated using the provisional execution information and/or higher than a predetermined threshold.

### (Supplementary Note 8)

The lifestyle habit recommendation apparatus according to any one of Supplementary Notes 5 to 7, wherein

the recommended information determination unit calculates the recommended execution information by changing the provisional execution information so that an evaluation based on a preset evaluation criteria can be raised.

### (Supplementary Note 9)

The lifestyle habit recommendation apparatus according to any one of Supplementary Notes 1 to 8, wherein
the lifestyle habit is a sleep habit.

### (Supplementary Note 10)

A lifestyle habit recommendation method, including the steps of:

acquiring execution information;

calculating a vitality level;

determining recommended information; and

outputting, wherein the execution information-acquiring acquires execution information executed for each item serving as an indicator of a lifestyle habit and a vitality level of a user in association with a date,

the vitality level-calculating calculates, based on the execution information and the vitality level of the user in a predictive input period, the vitality level of the user after a predetermined number of days from the predictive input period,

the recommended information-determining calculates recommended execution information up to the predetermined number of days so that the calculated vitality level can be raised, and

the outputting outputs the recommended execution information to the user.

### (Supplementary Note 11)

The lifestyle habit recommendation method according to Supplementary Note 10, wherein

the recommended information-determining calculates the recommended execution information when the calculated vitality level is low.

### (Supplementary Note 12)

The lifestyle habit recommendation method according to Supplementary Note 10 or 11, wherein

the vitality level-calculating calculates the vitality level of the user using sample information including the execution information and the vitality level acquired in association with the date.

### (Supplementary Note 13)

The lifestyle habit recommendation method according to any one of Supplementary Notes 10 to 12, wherein

the vitality level-calculating calculates the vitality level of the user using a learned model in which the execution information and the vitality level in the predictive input period are regarded as input and the vitality level after a predetermined number of days from the predictive input period is regarded as output.

### (Supplementary Note 14)

The lifestyle habit recommendation method according to any one of Supplementary Notes 10 to 13, wherein

the vitality level-calculating further acquires provisional execution information of the user up to a predetermined number of days after the predictive input period, and calculates the vitality level of the user based on the provisional execution information and the execution information and the vitality level in the predictive input period.

### (Supplementary Note 15)

The lifestyle habit recommendation method according to Supplementary Note 14, wherein

the vitality level-calculating calculates the provisional execution information based on the execution information in the predictive input period.

### (Supplementary Note 16)

The lifestyle habit recommendation method according to Supplementary Note 14 or 15, wherein

the vitality level-calculating further calculates the vitality level of the user based on post-change provisional execution information and the execution information and the vitality level in the predictive input period,

the post-change provisional execution information is obtained by changing a content of the item serving as an indicator of a lifestyle habit in the provisional execution information, and

the recommended information-determining calculates the post-change provisional execution information as the recommended execution information when the vitality level calculated using the post-change provisional execution information is higher than the vitality level calculated using the provisional execution information and/or higher than a predetermined threshold.

### (Supplementary Note 17)

The lifestyle habit recommendation method according to any one of Supplementary Notes 14 to 16, wherein

the recommended information-determining calculates the recommended execution information by changing the provisional execution information so that an evaluation based on a preset evaluation criteria can be raised.

### (Supplementary Note 18)

The lifestyle habit recommendation method according to any one of Supplementary Notes 10 to 17, wherein

the lifestyle habit is a sleep habit.

### (Supplementary Note 19)

A program for causing a computer to execute the method according to any of Supplementary Notes 10 to 18.

### (Supplementary Note 20)

A computer readable recording medium with the program according to Supplementary Note 19.

### INDUSTRIAL APPLICABILITY

According to the present invention, it is possible to provide a system which predicts a vitality level after a predetermined number of days and recommends an optimum lifestyle habit so that the vitality level can be raised. For this reason, the present invention is useful, for example, in the field of health care and the like.

### REFERENCE SIGNS LIST

10: lifestyle habit recommendation apparatus
11: execution information acquisition unit
12: vitality level calculation unit
13: recommended information determination unit
14: output unit

1. A lifestyle habit recommendation apparatus, comprising:
an execution information acquisition unit;
a vitality level calculation unit;
a recommended information determination unit; and
an output unit, wherein
the execution information acquisition unit acquires execution information executed for each item serving as an indicator of a lifestyle habit and a vitality level of a user in association with a date,
the vitality level calculation unit calculates, based on the execution information and the vitality level of the user in a predictive input period, the vitality level of the user after a predetermined number of days from the predictive input period,
the recommended information determination unit calculates recommended execution information up to the predetermined number of days so that the calculated vitality level can be raised, and
the output unit outputs the recommended execution information to the user.

2. The lifestyle habit recommendation apparatus according to claim 1, wherein
the recommended information determination unit calculates the recommended execution information when the calculated vitality level is low.

3. The lifestyle habit recommendation apparatus according to claim 1, wherein
the vitality level calculation unit calculates the vitality level of the user using sample information including the execution information and the vitality level acquired in association with the date.

4. The lifestyle habit recommendation apparatus according to claim 1, wherein
the vitality level calculation unit calculates the vitality level of the user using a learned model in which the execution information and the vitality level in the predictive input period are regarded as input and the vitality level after a predetermined number of days from the predictive input period is regarded as output.

5. The lifestyle habit recommendation apparatus according to claim 1, wherein
the vitality level calculation unit further acquires provisional execution information of the user up to a predetermined number of days after the predictive input period, and calculates the vitality level of the user based on the provisional execution information and the execution information and the vitality level in the predictive input period.

6. The lifestyle habit recommendation apparatus according to claim 5, wherein
the vitality level calculation unit calculates the provisional execution information based on the execution information in the predictive input period.

7. The lifestyle habit recommendation apparatus according to claim 5, wherein
the vitality level calculation unit further calculates the vitality level of the user based on post-change provisional execution information and the execution information and the vitality level in the predictive input period,
the post-change provisional execution information is obtained by changing a content of the item serving as an indicator of a lifestyle habit in the provisional execution information, and
the recommended information determination unit calculates the post-change provisional execution information as the recommended execution information when the vitality level calculated using the post-change provisional execution information is higher than the vitality level calculated using the provisional execution information and/or higher than a predetermined threshold.

8. The lifestyle habit recommendation apparatus according to claim 5, wherein
the recommended information determination unit calculates the recommended execution information by changing the provisional execution information so that an evaluation based on a preset evaluation criteria can be raised.

9. The lifestyle habit recommendation apparatus according to claim 1, wherein the lifestyle habit is a sleep habit.

10. A lifestyle habit recommendation method, comprising the steps of:
acquiring execution information;
calculating a vitality level;
determining recommended information; and
outputting, wherein
the execution information-acquiring acquires execution information executed for each item serving as an indicator of a lifestyle habit and a vitality level of a user in association with a date,
the vitality level-calculating calculates, based on the execution information and the vitality level of the user in a predictive input period, the vitality level of the user after a predetermined number of days from the predictive input period,
the recommended information-determining calculates recommended execution information up to the predetermined number of days so that the calculated vitality level can be raised, and
the outputting outputs the recommended execution information to the user.

11. The lifestyle habit recommendation method according to claim 10, wherein
the recommended information-determining calculates the recommended execution information when the calculated vitality level is low.

12. The lifestyle habit recommendation method according to claim 10, wherein
the vitality level-calculating calculates the vitality level of the user using sample information including the execution information and the vitality level acquired in association with the date.

13. The lifestyle habit recommendation method according to claim 10, wherein
the vitality level-calculating calculates the vitality level of the user using a learned model in which the execution information and the vitality level in the predictive input period are regarded as input and the vitality level after a predetermined number of days from the predictive input period is regarded as output.

14. The lifestyle habit recommendation method according to claim 10, wherein
the vitality level-calculating further acquires provisional execution information of the user up to a predetermined number of days after the predictive input period, and calculates the vitality level of the user based on the provisional execution information and the execution information and the vitality level in the predictive input period.

15. The lifestyle habit recommendation method according to claim 14, wherein
the vitality level-calculating calculates the provisional execution information based on the execution information in the predictive input period.

16. The lifestyle habit recommendation method according to claim 14, wherein
the vitality level-calculating further calculates the vitality level of the user based on post-change provisional execution information and the execution information and the vitality level in the predictive input period,
the post-change provisional execution information is obtained by changing a content of the item serving as an indicator of a lifestyle habit in the provisional execution information, and
the recommended information-determining calculates the post-change provisional execution information as the recommended execution information when the vitality level calculated using the post-change provisional execution information is higher than the vitality level calculated using the provisional execution information and/or higher than a predetermined threshold.

17. The lifestyle habit recommendation method according to claim 14, wherein
the recommended information-determining calculates the recommended execution information by changing the provisional execution information so that an evaluation based on a preset evaluation criteria can be raised.

18. The lifestyle habit recommendation method according to claim 10, wherein
the lifestyle habit is a sleep habit.

19. A computer readable recording medium with a program for causing a computer to execute the method according to claim 10.
